(19) United States
(12) Patent Application Publication
(10) Pub. No.: US 2019/0374677 A1
(43) Pub. Date: Dec. 12, 2019

(54) MODIFIED SURFACES FOR ATTACHMENT OF BIOLOGICAL MATERIALS

(71) Applicant: NORTHEASTERN UNIVERSITY, Boston, MA (US)

(72) Inventors: DANIEL M. STOREY, LONGMONT, CO (US); TERRENCE S. MCGRATH, LA JOLLA, CA (US); ALEXANDER B. REISING, ZIONSVILLE, IN (US)

(21) Appl. No.: 16/532,545

(22) Filed: Aug. 6, 2019

Related U.S. Application Data
(63) Continuation of application No. 15/399,778, filed on Jan. 6, 2017.
Application No. 15/399,778 is a division of application No. 11/603,436, filed on Nov. 20, 2006, now abandoned.
(60) Provisional application No. 60/786,118, filed on Mar. 27, 2006.

(51) Int. Cl. A61L 27/30; A61L 27/18; A61L 27/16; C12N 5/00; A61F 2/30

(52) U.S. Cl. CPC A61L 27/306; A61L 27/18; A61L 27/16; A61M 27/002; A61F 2/30767; A61M 2205/04; C12N 5/0068

(57) ABSTRACT

The invention relates to bioactive surface coatings deposited on selected substrates. Surface nanostructured film coatings deposited on most metal or nonmetal substrates to provide surfaces can be engineered to promote enhanced tissue/cell adhesion. Attached cells, including osteoblasts, fibroblasts and endothelial cells, retain viability and will readily differentiate and proliferate under appropriate conditions. Fibroblasts and endothelial cells exhibit good attachment and growth on most coated substrates, except on nano surfaced structured silicone.

This application is a continuation of U.S. application Ser. No. 15/399,778, filed Jan. 6, 2017, which is a divisional of U.S. application Ser. No. 11/603,436, filed Nov. 20, 2006, which claims benefit of provisional application Ser. No. 60/786,118, filed Mar. 27, 2006, the entire contents of which are herein incorporated by reference.

### BACKGROUND OF THE INVENTION

### 1. Field of the Invention

The invention relates to modified coatings that provide an adhesion matrix for cells and other biological materials. Selected nano-textured coating surfaces promote cell growth and proliferation and can be deposited as stable coatings on metal or non-metal substrates.

### 2. Description of Background Art

Rejection of implantable devices is a major problem because the body does not recognize foreign materials as self. Consequently, a wide range of medical devices in current use fail to promote healing and may often promote a high infection rate. Catheters, joint replacements, soft tissue repair and dental implants are examples where these problems are frequently observed. Implants should ideally promote cell attachment so that infection is minimized and healing rate is increased.

The materials from which implant devices are made are usually surface modified in attempts to promote or improve cell attachment. Unfortunately, even when there is cell attachment, there are no long term indwelling devices that adequately promote tissue growth. Some protocols employ growth factors on the surface of a device to aid in tissue attachment, but results are not always satisfactory and growth factors are not routinely used. Few successful attempts have been made to modify implant surfaces so that new tissue cells readily attach and grow, and current technology has failed to develop surfaces that significantly enhance tissue attachment.

Some attention has been paid to bioactive coatings that improve the performance of conventional titanium-based materials for orthopedic applications. Devices in current use are fabricated by traditional metallurgy techniques by applying hydroxyapatite as a surface coating over titanium in an effort to enhance bone attachment. Commercially, hydroxyapetite is coated on titanium-based metals by a high-temperature plasma-spray deposition process, which transforms nanocrystalline hydroxyapatite into micron grain size hydroxyapatite containing a less crystalline calcium phosphate matrix. Plasma spray deposition of hydroxyapatite is one coating method that has been used; however, this results in phase transitions that may lead to the formation of highly soluble calcium phosphates, which cause delamination of the coating during clinical use (Furlong, et al., 2001; Baker, et al., 2006).

Recently, techniques have been explored for obtaining surface roughness on a nanoscale, including the use of ultrafine metal coatings such as titanium (Webster et al., 2004; Valiev, et al., 2004). Anodized titanium and chemical etching of deposited titanium have also been used in attempts to create surfaces attractive for osteoblast attachment and subsequent bone formation (Yao, et al., 2005).

Materials with nanometer surface features are thought to enhance bone formation compared to materials with micron scale features (Sato, et al., 2005; Popat, et al., 2005). Unfortunately, for currently used implants, conventional coating processes do not provide the nanostructured surfaces required for effective bone regeneration.

The majority of current efforts aimed toward enhancement of tissue attachment to implantable devices have focused on developing pressed metal implants constructed from nano powders and to nano texturing of plastics. The problem with both methods is that the materials lose a significant amount of strength because of surface modifications.

A recent approach to the design of next-generation orthopedic implants has centered on matching synthetic implant surfaces to the unique nanometer topography created by natural extracellular matrix proteins found in bone tissue. While the nanometer structures and molecules found in bone tissue show that bone-forming cells typically interact with surfaces of nanometer roughness, conventional synthetic metals currently in use have micro-rough surfaces but are smooth at the nanoscale level (Kaplan, et. al., 1994A; Kaplan, et. al. 1994B).

Woven (or immature) bone has an average inorganic mineral grain size of 10-50 nm. Lamellar bone, which actively replaces woven bone, has an average inorganic mineral grain size of 20-50 nm long and is 2-5 nm in diameter. However, at nano-scale dimensions, many, if not all, currently utilized implant surfaces are smooth. Such smooth surfaces have been shown to favor “fibrointegration,” (callus formation) which can ultimately encapsulate implants placed in bone with stratified undesirable connective tissue (Webster, et al., 2004).

In addition to efforts to develop cellular attachment coatings on orthopedic devices, there is a need for attachment coatings on devices such as those used for dental implants. Hydroxyapatite or ACTIPORE™ coatings are not easily deposited on typical medical device substrates such as CoCrMo. Even when deposition is possible, adhesion is often poor and delamination can occur.

There are two main technologies currently used to make surfaces that promote tissue attachment. One method is to press metallic nano-powders into forms so that some surface roughness is obtained; the other method is to create nano-rough surfaces on plastics through a molding process.

The pressing of metallic powders into forms and sintering at a low temperature creates a surface that promotes ingrowth of tissue on a substrate surface. Unfortunately, such compositions cannot be used in many orthopedic applications because the strength required for orthopedic use requires the powder to be sintered at a high temperature in order to obtain the necessary strength. The elevated sintering temperature destroys the micro-structure of the surface and thus any advantage for tissue attachment is lost.

Molding nano-texturing into polymer surfaces has been the main thrust of efforts to design surfaces that promote tissue growth. This method has met with limited success, in part because the mold has only a limited ability impart the correct nano-texturing to a plastic surface with consistent results. Quality control in the manufacturing process is generally unacceptable because plastic flow into a rough mold is difficult to control and part rejection rates may run as high as 50%.

### DEFICIENCIES IN THE ART

The deficiencies in surface pressing and molding techniques indicate the need for methods to produce surface coatings that have much improved tissue adherence properties and are appropriate for use as coatings on medical implants.

Accordingly, there is a need for coatings that adhere to metal or nonmetal surfaces, have superior tissue attachment properties and are nontoxic to living cells. Attachment coatings with these characteristics would ideally be deposited on a wide range of substrate surfaces in a consistent and economically viable process.

### SUMMARY OF THE INVENTION

The present invention is based in part on the recognition that nano-particle (particles up to no larger than about 100 nm) deposition can be precisely controlled, and the unexpected discovery that coatings deposited by a modified ion deposition process (IPD) on selected substrates enhance tissue attachment to a significantly greater extent than coatings deposited by conventional plasma vapor deposition methods. This observation has resulted in the development of a method for producing nanostructured coatings that act as biocompatible scaffolds for bone regeneration.

A particularly surprising discovery was the observation that IPD deposited metal coatings on silicone, in contrast to IPD deposited metals on other metal and polymer based substrates, do not promote attachment of some cell types; for example fibroblasts or endothelial cells. Most cells tested, however, exhibited enhanced attachment and proliferation on IPD deposited metal surfaces on metal or several different types of polymers. Use of silicone substrates may be highly advantageous when selective osteoblast adherence is desired in order to promote bone growth, because fibroblasts promote soft tissue and callus formation. Bone regeneration may be comprised on implants intended to promote bone growth and may delay or inhibit recovery.

It has been found that several types of cells readily attach to the nanostructured coatings and that the attached cells will proliferate in appropriate environments. Attached immature cells, such as fibroblasts and osteoblasts, will differentiate and proliferate on a nano-structured support coating, which acts as a scaffolding or matrix. As discussed, an exception is attachment of fibroblast or endothelial cells when nanostructured IPD deposited metal surfaces are coated on silicone substrates.

The IPD method used to produce the nanostructured coatings is based on a modified IPD process tuned to increase nano-particle production and control deposition. The IPD deposited metal can be deposited from a controlled speed plasma arc target at a preferred switching rate of about 300 Hz to 500 Hz to obtain the desired nanostructured coatings.

It has been discovered that using a controlled IPD process for depositing metals on plastic or metal substrates enhances adhesion of nano-particle materials to the substrate surface. The deposited coatings promote higher tissue cell adhesion rates than films or surfaces produced by other processes used in the industry. The IPD process deposits nano-particle materials directly onto the substrates without need for special or primer “seed” coatings.

Tight control of nanoparticle size and density of deposited material on a substrate surface initially was not expected to improve cell adhesion and tissue growth on implanted devices. The predominant trend for persons skilled in plasma deposition processes for years has been to reduce the number of nanoparticles deposited on surfaces in order to produce cleaner and more uniform films. Conventional wisdom in the industry was that particles even in the 1-micron size range in general are deleterious to the quality of deposited films so that deposited films should be as smooth and particle-free as possible.

Thus, it was contrary to expectations that nano-sized particles less than about 100 nm in size ejected from a target and deposited on a substrate actually enhanced the tissue attachment quality of a coating rather than diminished it. An important aspect of the invention is therefore the development of methods for increasing rather than reducing nano-particle production and controlling the size of the nano-particles. While it is generally known that ion plasma deposition processes can achieve higher deposition rates and tend to produce more macro-particles than other types of plasma vapor deposition (PVD) processes, it was not previously known or appreciated that more, not less, nano-particle deposition would enhance tissue attachment properties of deposited nanostructured surfaces.

Consequently, efforts by others to improve plasma arc deposition methods and apparatus by focusing on reducing rather than increasing macro-particle production have met with little success in improving tissue attachment. The results described herein demonstrate that depositing films with ultra nanoparticle density significantly improves tissue attachment characteristics of IPD produced thin films and that such films are particularly advantageous for use on implant devices. Macro particles in a selected nanoparticle size range, which are generally seen as not useful for performance improvements, can be purposely produced to enhance tissue attachment coatings.

Methods have been developed that are particularly well-suited to the rapid deposition of nano-textured coatings, not only for depositing at high rates to achieve better adhesion, but also to increase nano-particle deposition. Accordingly, the invention includes a method for enhancing production and deposition of nano-particle dense coatings of bio-compatible materials. The coatings exhibit improved tissue attachment and adhesion characteristics. The result of using a modified IPD nano-particle coating process is a dense, highly conformed, highly adherent, thick coating, which is well suited for promoting tissue attachment on implanted medical devices used in human and veterinary applications.

The coatings produced by the IPD method are not limited by the type of substrate and can be applied to a wide range of materials, including non-conductive materials such as plastics and ceramics and conductive materials such as metals. The method of creating a controlled nano-textured surface can be used to deposit biocompatible films on medical devices, which accelerate healing at implant sites.

It is therefore an object of the present invention to provide a method of depositing attachment coatings onto a substrate using a modified IPD process to form controlled nano-dense tissue attachment coating surfaces.

The coatings are produced using a modified IPD deposition of an attachment surface on a substrate. A target comprising a potential attachment metal or combination of metals is placed in an evacuated chamber and the target is powered to generate an arc which ionizes the target metal into a plasma of ionized particles. A reactive gas such as oxygen or nitrogen is optionally introduced into a vacuum chamber so that the gas reacts with the ionized plasma particles. Deposition of the plasma particles onto the substrate is controlled by variably controlling the power to the target and/or optionally moving the substrate closer or further from the target in a controlled manner during the deposition process.

The IPD method provides an attachment surface on medical devices or materials, which promotes faster healing in vivo than is provided by conventional medical devices and materials, whether or not conventionally coated. This is accomplished by depositing a metal on a polymer or metal substrate so that a highly conformed nanostructured surface is formed on the substrate.

Dispersed metal, metal nitride or metal oxide particles can be deposited by IPD on a wide variety of substrate materials, including metal, plastic, glass, flexible sheets, porous papers, ceramics, combinations thereof and the like. While the substrate may comprise any of a number of devices, medical devices are particularly preferred and may include catheters, implants, stents, tracheal tubes, orthopedic pins shunts, drains, prosthetic devices, dental implants, dressings and wound closures. It should be understood that the invention is not limited to such devices and may extend to other devices useful in the medical field, such as face masks, clothing, surgical tools and surfaces.

The target may be any solid material or combination of materials having attachment properties, provided that the target material is capable of ionization via an arc plasma process. Preferred materials are metals having potential attachment properties and which are biocompatible; i.e., not damaging in the intended environment. Such materials include alloys and metals, including zinc, niobium, tantalum, hafnium, zirconium, nitinol, titanium, titanium 6-4, chromium, cobalt, nickel, copper, molybdenum, iron/chromium/nickel (stainless steel), platinum and gold, referred to herein generally as “attachment metals.”

The present invention provides the deposition, impregnation or layering of gold, titanium, nitinol or other metal ions onto a substrate surface to form a dense nanostructure comprised of particles greater than 5 nm. The nanostructured surface provides attachment points for cells or other biological materials. Cells become bound onto the solid state structures of nano-pico and micro-sized crystalline metal and metal oxide compounds, which may deposit as combinations of mono, di-, and polyvalent oxides dispersed into or onto a surface.

In general, the invention is directed to preparing a biocoated substrate, comprising depositing a metal ion plasma on a substrate to form a nano-structured densely distributed particulate metal coating and contacting the coating with one or more cells for a time sufficient to attach the one or more cells to the coating surface. The one or more cells attached to the deposited coating form a biocoated substrate that retains biological properties of the attached cells. In effect, the biocoating is attached to a matrix or scaffolding that allows cells or tissues to readily attach and grow under appropriate conditions, whether in an artificial culture environment or in a natural environment, as might be the case for a medical implant. Where immature cells attach, the biocoat may allow differentiation, e.g., maturation of osteoblasts into bone cells.

Virtually any cell may be attached to the nanotextured surface coating; in general any mononuclear cell. Examples include leucocytes, lymphocytes, neutrophils, eosinophils, monocytes and the like. Particularly preferred cells include osteoblasts, fibroblasts and endothelial cells. Mixtures of different cells are also expected to be able to readily attach to these cells and be able to grow and proliferate.

The biocoatings are produced on various substrate surfaces using an ion plasma deposition (IPD) process. A metal selected as the coating material acts as a target which produces metal ions that deposit on an anode target when an ionized beam or arc is produced between the target and the substrate anode. When the production of metal ions at the target is controlled by managing arc speed and deposition at the anode substrate is controlled by its relative distance from the target, it is possible to create highly dense nanoparticulate surfaces. These nanoparticles are embedded into the substrate surface so that they are stable and highly resistant to peeling. Importantly, they act as a cell-friendly matrix, making them ideal for coatings on medical implants.

The IPD deposited metal ions are preferably densely deposited as nanoparticles, not as larger particles approaching micro size. The most preferable size range for nanoparticle size is about 1 to about 100 nanometers with about 15 nm being particularly preferred for titanium and gold, which are two of the more popular coating metals. Nanoparticle densities of about 10³ particles/cm² to about 10⁴ particles/cm² are typical densities that provide good biocoats. Thickness of the coating is preferably about 0.1 to about 3 microns.

Targets for the IPD method can be any metal, although in consideration for use in or on living organisms, nitinol, CoCrMo, gold, platinum, copper, tantalum, titanium, zirconium, hafnium, zinc or combinations thereof are preferred with nitinol, gold and titanium being particularly preferred.

Substrates can be any of a number of materials, whether metal or non-metal including plastics and ceramics. Exemplary substrate materials include UHMWPE, EPTFE, PTFE, PEEK, polypropylene, polyurethane, polyimide, polyester, nylon, titanium, iron/chromium/nickel (steel), cobalt, chromium, zirconium, nickel, nitinol, alloys and combinations thereof.

The invention also includes compositions comprising one or more bioviable cells attached to a nano-structured metal film. The film is produced from ion plasma deposited metal particles that are about 1 micron in size distributed at a density of about 10³ to 10⁴/cm². Typical metals deposited include Ag, Au, Ti, CoCrMo, and mixtures thereof.

The bioviable cells can be any mononuclear cell. Particularly preferred cells are those that may be in contact with surface coated medical devices such as implants where fibroblasts, osteoblasts or endothelial cells are most likely to be present.

A preferred embodiment includes osteoblast cells attached to a nano-structured titanium surface deposited on UHMWPE. A preferred surface for endothelial cells is titanium deposited on UHMWPE or PTFE. The metal surface coating will generally comprise particles up to 15 nm in size distributed on the substrate surface at a density of about 10³ to about 10⁴/cm² and having a thickness of about 0.3 to about 1 nm. Alternative nano-textured metal surfaces include gold, titanium and nitinol.

The nano-structured surface coatings produced by the IPD method are highly stable because the coating impregnates a metal or polymer substrate up to a depth of about 10 to about 100 nanometers. An exemplary preferred ion plasma deposited metal surface can be comprised of nanoparticulates about 1 to about 100 microns in size, at a surface density of about 10³ to 10⁴/cm² and a thickness of about 10 to 100 microns.

### Definitions

Ionic Plasma Deposition (IPD) is a method of creating highly energized plasma using a cathodic arc discharge in a target material, typically solid metal. An arc is struck on the metal and the high power density on the arc vaporizes and ionizes the metal, creating a plasma which sustains the arc. A vacuum arc is different from a high pressure arc because the metal vapor itself is ionized, rather than an ambient gas.

Plasma vapor deposition (PVD) is a thin film deposition process in the gas phase in which source material is physically transferred in the vacuum to the substrate without any chemical reaction involved. This type of deposition includes thermal evaporation, electron beam deposition and sputtering deposition. The IPD process is a subtype of physical vapor deposition.

Macros or macro particles are descriptive of particles ejected from a target and as used herein will refer to particles larger than about 100 nm while nano particles are particles up to about 100 nanometers in size.

“Attachment properties” and “potential attachment properties” are terms intended to recognize the fact that some metals, in their elemental state, are typically too unreactive to act as effective attachment sites, but may exhibit a much stronger attachment effect when ionized. Thus the attachment metals comprising a target have potential attachment properties which in many cases are realized upon ionization of the metals. When ionized, the attachment metals can also be combined with various reactive gases such as oxygen or nitrogen to form oxides or nitrides and combinations thereof.

Biological materials as used herein include tissue components such as cells, mineralization inorganic substances such as hydroxyapatite and biological matrix material, such as collagen.

Nitinol, unless otherwise indicated, is defined as an approximately 55/45 combination of nickel and titanium respectively with a specified grain structure.

The term “about” as used herein is intended to indicate that a specified number is not necessarily exact but may be higher or lower within a 10% range as determined by the particular procedure or method used.

The term “a” as used in the claims is not intended to limit to a single species.

Accepted abbreviations for several polymers include: PEEK (polyether ether ketone); PTFE (poly tetrafluoroethylene); EPTFE (expanded poly tetrafluoroethylene); and UHMWPE (ultra high molecular weight polyethylene).

CoCrMo is an alloy of cobalt, chrome and molybdenum typically in the ratio of about 64%, 28% and 6% respectively; Ti-gal-4V is an alloy used in surgical implants containing 89% titanium, 6% aluminum and 4% vanadium.

KSI is a standard pull test which applies 1000 psi to a surface to test for adhesion.

As used herein, “bioviable” is a descriptive term indicating that a biological material maintains its natural biological potential; for cells this means maintaining growth and proliferative capacity.

Biocoats are films adhered to a base material or “substrate”, which have properties of biological materials, e.g., cells, tissues, cell matrices and inorganic structural components such as hydroxyapatite and bone.

### BRIEF DESCRIPTION OF THE DRAWINGS

FIG. 1 shows the general features of a modified cathodic arc IPD apparatus: target 1; substrate 2; movable substrate holder 3; vacuum chamber 4; power supply 5 for the target; and arc control 6 to adjust speed of the arc.

FIG. 2 shows a comparison of a titanium coated substrate (titanium) with the uncoated substrate. Deposition of the titanium results in a nanoscale surface roughness as can be seen in the low magnification SEM photographs of uncoated and IPD deposited titanium on UHM and PTFE. Bars=10 microns for both uncoated samples, 20 microns for UHMWPE coated with titanium and 10 microns for PTFE coated with titanium.

FIG. 3A shows increased osteoblast density on UHMWPE and PTFE coated with Ti after 1 day for N=3, *P<0.01 and **P<0.01 compared to density of cells attaching to a titanium metal bar.

FIG. 3B shows increased osteoblast density on UHMWPE and PTFE coated with Ti after 3 days for N=3, *P<0.01 and **P<0.01 compared to density of cells attaching to a titanium metal bar.

FIG. 3C shows increased osteoblast density on UHMWPE and PTFE coated with Ti after 5 days for N=3, *P<0.01 and **P<0.01 compared to density of cells attaching to a titanium metal bar.

FIG. 4 compares fluorescent microscopy images of increased osteoblast density on uncoated PTFE and PTFE coated with Ti after 1, 3 and 5 days. Bars represent 100 microns.

FIG. 5 shows increased osteoblast (calcification) formation on solid titanium metal, UHMWPE, PTFE, coated UHMWPE, and coated PTFE after 7, 14 and 21 days. N=3 samples, *p<0.01 compared to the corresponding uncoated sample and **p<0.01 compared to a solid titanium metal bar.

FIG. 6 shows cell adhesion for Ti coated silicone, polyethylene and Teflon® for N=3; *p<0.01 compared to respective uncoated samples.

FIG. 7 shows fluorescent microscopy images of coated and uncoated silicone, polyethylene and Teflon® comparing differences in cell counts on the different surfaces.

FIG. 8 shows fibroblast adhesion comparisons for titanium coated UHMWPE and PTFE compared to the respective uncoated samples. Also shown in the figure is decreased cell adhesion of fibroblasts on titanium coated silicone compared with uncoated silicone. Data are averages of three samples, n=3, where *represents p<0.01 compared with the uncoated counterparts.

FIG. 9A is a graph comparing fibroblast proliferation on titanium coated silicone, UHMWPE and PTFE with respective uncoated samples after 1 day compared with respective uncoated samples. Each bar represents the average of 3 samples; ⁺p<0.01 compared to the uncoated substrates.

FIG. 9B is a graph comparing fibroblast proliferation on titanium coated silicone, UHMWPE and PTFE with respective uncoated samples after 3 days compared with respective uncoated samples. Each bar represents the average of 3 samples; ⁺p<0.01 compared to the uncoated substrates.

FIG. 9C is a graph comparing fibroblast proliferation on titanium coated silicone, UHMWPE and PTFE with respective uncoated samples after 3 days compared with respective uncoated samples. Each bar represents the average of 3 samples; where *p<0.01 compared to the uncoated substrates.

FIG. 10 shows fluorescent images of titanium coated silicone, polyethylene and Teflon® substrates showing the differences between numbers of fibroblast cells on these surfaces compared to the uncoated substrates.

FIG. 11 is a bar graph showing changes in protein levels as measured by absorbance after 7, 14 and 21 days for titanium coated silicone, UHMWPE, and PTFE samples compared with uncoated samples. *p<0.01 compared to the respective uncoated sample and previous time point.

FIG. 12A shows osteoblast proliferation after 1 day comparing titanium coated silicone, UHMWPE and PTFE with the respective uncoated substrates. Each bar represents the average of three samples; *p<0.01.

FIG. 12B shows osteoblast proliferation after 3 days comparing titanium coated silicone, UHMWPE and PTFE with the respective uncoated substrates. Each bar represents the average of three samples; *p<0.01.

FIG. 12C shows osteoblast proliferation after 5 days comparing titanium coated silicone, UHMWPE and PTFE with the respective uncoated substrates. Each bar represents the average of three samples; *p<0.01.

FIG. 13 is a photo panel comparing fluorescent images of osteoblast proliferation on titanium coated and uncoated PTFE after 1, 3 and 5 days.

### DETAILED DESCRIPTION OF THE INVENTION

The present invention provides a number of advantages over other state of the art attachment coatings and processes for depositing attachment coatings. The IPD deposition method used to prepare the improved bio-coatings enables control of particle size, lower run temperatures for certain materials, significantly improved throughput processing efficiency compared with conventional plasma arc processes, scalability and application to a wide range of substrate materials. An important characteristic of the deposited material is high surface adherence to the substrate, due in part to embedding of the ionized particles in the substrate surface. The IPD deposited surfaces comprise densely arranged nanoparticles which contribute to the surface features that significantly enhance cell/tissue attachment, differentiation and proliferation.

The disclosed IPD process is performed under vacuum and is used to produce the nanostructured surfaces that promote cell attachment. Typical energy levels of 150 eV to 500 eV are controlled appropriately, depending on the target material, which is preferably nickel, titanium, gold and/or alloys or compositions containing these metals. Energy levels also depend on the size of the target, so that where the target is large, higher energy input may be required. The process allows deposition at temperatures at least as low as about 30° C., which is a preferred temperature range for deposition on thermosensitive resin and plastic substrates.

In general, the method requires positioning a selected substrate between a target and an anode housed within a vacuum chamber, said target comprising an ionizable metal. An arc discharge is generated between the target and the anode. Power to the target is variably controlled so that macro particles having a size of about 100 nanometers to about 5 microns are produced. Optionally, or in addition, one may adjust movement of the substrate within a range of about 10 inches to about 30 inches toward or away from the target for a predetermined time at a temperature of between about 25° C. and about 75° C. during arc discharge. This will produce a high density, macroparticulate, adherent attachment coating film having a thickness of about 1 nm to about 50 microns on the substrate.

Superior coatings unavailable using conventional vacuum arc deposition (VAD) methods have been obtained, including surfaces coated with exotic nickel/titanium alloys, exotic CoCrMo alloys and other alloys not usually considered as coatings for use in medical devices or applications. Thinner coatings and shorter processing time can be achieved with the same or better attachment affinities when the modified IPD-based process is employed. Higher throughput is possible, which can result in production cost savings and is a significant advantage, particularly in the medical industry.

In accordance with the disclosed method, attachment metals are deposited onto or into the surface of a substrate by ionizing a target metal into a plasma. There are many ionic plasma deposition devices, such as those described in International publication WO 03-044240, the contents of which are herein incorporated by reference. These basic devices can be modified and used to carry out the controlled deposition of selected metals for use as coatings suitable for cell attachment.

When depositing a coating on a substrate, the relative number of macro particles ejected from the target can be controlled. Macro particles are molten blobs of metal that are ejected from the target without being completely vaporized. The blobs are dense and comprised of pure target material. The blob surface is usually charged, while the bulk of the material is neutral.

An important feature of the modified IPD process is the ability to imbed a metal or metal/oxide coating into a substrate surface, thus obtaining superior adhesion compared to coatings deposited by other deposition methods. The imbedding process can be controlled by adjusting the arc at a specific distance from the target. Coatings embedded up to at least 100 nm for plastics and up to at least 10 nm for metal and ceramic substrates can be obtained.

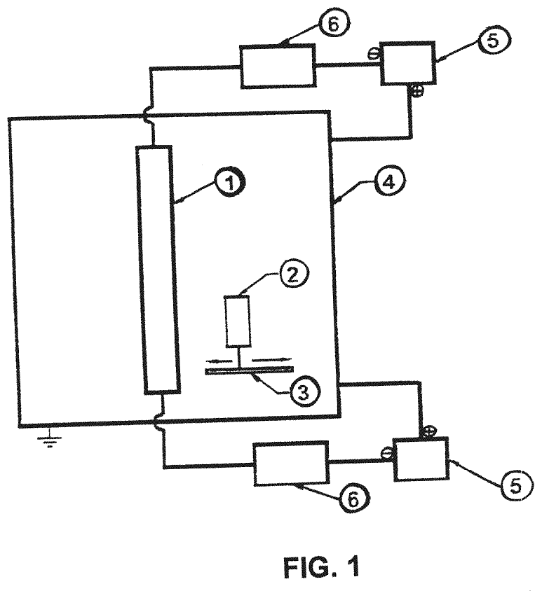

A suitable device for carrying out a modified plasma arc deposition process is the IPD process illustrated in FIG. 1. As shown in FIG. 1, a cathode of the target material 1 is disposed within a vacuum chamber 4. The target is ionized by generating an arc at the target from a power supplied by a power source 5. The plasma constituents are selected, controlled or directed toward the substrate by a controlling mechanism 3 that moves the substrate 2 toward or away from the target. A power supply control 6 is used to control arc speed.

IPD is not necessarily a line of sight deposition method. While rotation and racking are necessary for complex geometries, the racking and rotation is usually not nearly as complex as it is for other PVD processes. In addition, this process produces a repeatable hole penetration aspect ratio of 5:1 for any sized hole over 10 micron. It is difficult to test a hole less than 10 microns due to macro particle accumulation.

Typical coating rates achieved with the IPD process in this invention range from about 100 nm to 5 microns per minute for materials such as gold or silver. Coating areas over 45,000 square inches per hour at a coating rate of greater than 200 nm per minute for these materials have been obtained. In addition to the increased coating rate and large volume, the IPD process requires less handling per square inch because only a single layer coating is required, which means lower labor and higher processing rates/throughput.

The effectiveness of the attachment response is also dependent upon the processing time for forming the attachment surface. Longer processing times from 5 seconds to several minutes result in attachment surfaces having different attachment responses.

Particle size of the IPD deposited coatings is preferably controlled by adjusting power to the target such that particle size is in the range of about 100 nanometers to about 5 microns, with particles in the nanometer range being preferred for coatings on medical devices where tissue attachment is desired. Titanium or gold particles deposited by the disclosed methods can be controlled to particles sizes less than 100 nm in diameter.

Surfaces coated using this IPD process are surprisingly compatible surfaces for cell proliferation and growth. A range of cell types will adhere to metal coated substrates and exhibit significantly enhanced growth compared to uncoated surfaces. Tissue growth enhancement on IPD deposited metals on nonmetal substrates has been demonstrated with osteoblasts, fibroblasts and endothelial cells. This has significant implications for use of these biocompatible coatings in medical applications such as hip replacements and other orthopedic implants.

While osteoblasts are known to at least initially adhere to gold or titanium coated polymers, IPD deposited gold or titanium on several types of polymers is shown her to significantly enhance adhesion and continued long term growth, being especially notable on titanium coated UHMWPE where cell adhesion increased was increased almost 600% after 5 days and was highly significant even after 21 days. Increased cell adhesion was also observed for gold or titanium coated PEEK and gold coated PTFE, although the latter showed relatively low adhesion for osteoblasts.

Similar effects were observed with endothelial cells on titanium coated UHMWPE where a 500% increase in cell adhesion was noted with a 100% increase on titanium coated PTFE compared with uncoated samples.

Fibroblasts appeared to follow the same pattern, with increases in cell density of 78% on titanium coated PTFE and 90% on UHMWPE compared with uncoated samples.

In sharp contrast to titanium coated silicone, fibroblasts showed markedly less tendency toward adhesion than silicone or titanium alone.

The invention is further illustrated by the following non-limiting examples.

Materials and Methods

Human Osteoblasts

Human osteoblasts (CRL-11372, American Type Culture Collection, population numbers 2-4) were used in the cell adhesion experiments in this study. All substrates of interest were rinsed with phosphate buffered saline (PBS) (1× strength) before seeding the cells. The cells were cultured on the substrates in Dulbecco's Modified Eagle Medium (Hyclone) supplemented with 10% fetal bovine serum (Hyclone) and 1% penicillin/streptomycin (Hyclone) with an initial seeding density of 3500 cells/cm² of substrate. Cells were then allowed to proliferate on the substrates under standard cell culture conditions (37° C. temperature, 5% CO₂ and 95% humidified air) for 1, 3 and 5 days; media was changed every other day. After the prescribed time period, the cell culture medium was aspirated from the wells and the substrates were gently rinsed with PBS three times to remove any non-adherent cells. The cells were then fixed with a 4% formaldehyde solution (Fisher) and stained with DAPI (Sigma). The cell numbers were counted and images taken under a fluorescence microscope (Swiss).

For long-term cell experiments, osteoblasts were seeded at a cell density of 50,000 cells/scaffold and were cultured in DMEM supplemented with 10% FBS, 1% P/S, 2.16×10⁻³ g/ml β-glycerophosphate, and 5×10⁻⁵ g/ml ascorbate for 7, 14, and 21 days. At the end of the prescribed time periods, cells were lysed using three freeze-thaw cycles. In order to determine the amount of calcium-containing mineral that had been deposited by osteoblasts, substrates were then soaked in 1 N hydrochloric acid (J. T. Baker) overnight to dissolve the calcium mineral deposits. These supernatants were then collected and tested for calcium content using a Calcium assay (Sigma Diagnostics; Procedure No. 587) following the manufacturer's instructions. All experiments were run in triplicate and repeated at least three different times.

Endothelial Cells

Rat aortic endothelial cells were purchased from Vec Technologies (Greenbush, N.Y.) and were grown to confluence in DMEM with 10% FBS and 1% P/S. Before cell experiments, samples were sonicated and autoclaved.

Endothelial cells were seeded onto each substrate at 3500 cells/cm². Samples were first placed in 12- and 24-well cell culture plates. 175 μl of cell-containing droplets in media was added to the wells and then incubated at 37° C. under 5% CO₂ for 4 hours. Specimens were washed 3 times with PBS, fixed in formaldehyde for 10 min, and again washed in PBS 3 times. Cells were counted using fluorescent microscopy and DAPI dye. Images of cell morphology were also obtained. Experiments were conducted in triplicate with each repeated twice (total of six samples for each averaged data point). A student t-test was used to determine differences between substrates.

Fibroblasts

Fibroblasts (CRL-2317, American Type Culture Collection, population numbers 2-4) and osteoblasts (CRL-11372, American Type Culture Collection, population numbers 2-4) were used in the cell experiments. Substrates were rinsed with phosphate buffered saline (PBS) (1X strength) before seeding the cells. The cells were cultured on the substrates in Dulbecco's Modified Eagle Medium (Hyclone) supplemented with 10% fetal bovine serum (Hyclone) and 1% penicillin/streptomycin (Hyclone) with an initial seeding density of 3500 cells/cm² of substrate. Some experiments were performed with fibroblasts alone and some by simultaneously seeding fibroblasts and osteoblasts (pre-stained with different fluorescent markers; Molecular Probes) to ascertain competitive cell adhesion. Cells were then allowed to adhere on the substrates under standard cell culture conditions (37° C. temperature, 5% CO₂ and 95% humidified air) for 4 hours. After the prescribed time period, the cell culture medium was aspirated from the wells and the substrates were gently rinsed with PBS three times to remove any non-adherent cells. The adherent cells were then fixed with a 4% formaldehyde solution (Fisher) and stained with a Hoescht 33258 dye (Sigma). The cell numbers were counted under a fluorescence microscope (Swiss).

Surface Characterization

Scanning electron microscope (SEM) analysis of IPD deposited coated surfaces was conducted using field emission scanning electron microscopy (LEO) JEOL JSM-840 Scanning Electron Microscope at a 5 kV accelerating voltage. Digital images were recorded using the Digital Scan Generator Plus (JEOL) software. Fluorescent microscopy images were obtained with a Leica fluorescence microscope, excitation wavelength at 365 nm and absorbance measured at 400 nm.

Statistics

Statistical analysis was performed using standard analysis of variance (ANOVA) techniques coupled with a Duncan's Multiple Range test. All experiments were run in triplicate with at least three replicates; p<0.01 was considered statistically significant.

### EXAMPLES

### Example 1-Controlled IPD Deposited Metal Films

FIG. 1 illustrates an apparatus suitable for controlling deposition of the plasma ejected from the cathodic arc target source (1) onto a selected substrate (2). The size of the particle deposited, and thus the degree of nanotexturing of the deposited surface is controlled by a movable substrate holder (3) within the vacuum chamber (4) or by a power supply (5) to the target and adjustment of arc speed (6). The closer a substrate is to the arc source, the larger and more densely packed will be the particles deposited on the substrate.

To prepare the coated substrates used for cell adhesion, a fairly macro-free film was deposited by positioning a substrate at a relatively far distance from the target. This formed an adhesive film. A more macro dense film was then deposited by positioning the substrate closer to the target.

Control of Substrate Distance from Target

Referring to FIG. 1, a substrate (sample 1) was placed in the movable substrate holder (3) at a distance of 30 inches from the target. The chamber (4) was pumped to a level of 5E-4 Torr. The arc was initiated with a current of 100 amps and 16 volts. The substrate (2) was translated closer to the target at a speed of one inch every 15 seconds and continued until the substrate was 8 inches from the target (30 min).

Substrate (sample 2) was placed at a distance of 30 inches from the target in a vacuum chamber pumped to a level of 5E-4 Torr. The arc was initiated with a current of 100 amps and 16 volts. The substrate was maintained at a distance of 30 inches from the target for 30 min.

Cross sections of sample 1 and sample 2 were examined using SEM analysis. In sample 1 the amount and size of macro particles increased with the thickness of the film; i.e., there were fewer and smaller macro particles close to the substrate, and the number and size increased as the thickness of the film grew. Conversely, the cross section in sample 2 was uniform with very few macro particles.

Control of Arc Power

Nano particle deposition and size can also be controlled by use of a controlled IPD power source, which can be configured to sufficiently slow or accelerate the speed of the arc. The traveling speed of the arc is directly related to the number of macro particles produced. Slowing the speed of the arc on the surface of the target causes it to produce more macro particles, which can be used to increase the macro particle density. The resulting increased film density also increases the ability of tissue to attach to the film. Conversely, increasing the speed of the arc on the target will decrease production of macro particles. This produces more high energy ions that can be embedded into the surface of the substrate to produce better adhesion.

Sample 3 had no arc control and the substrate was placed at a distance of 12 inches from the target. Both samples were placed in the chamber, at separate times for separate runs, and pumped to 5E-4 Torr. The arc was set at 100 amps for the power supplies. Each target had two supplies for a starting total of 200 amps. Sample 3 was run for five min with no arc control.

Sample 4 was run with an optimized switching of current at a rate of 300 Hertz.

Switching was controlled to maintain 200 amps on the target, but each power supply was ramped up or down so that at any time the current was not equal on the supplies. This forced the arc to travel a specific distance in a given amount of time, thereby controlling the macro particle density and size.

SEM cross sectional analysis was performed on samples 3 and 4. The films were consistent throughout the entire thickness except that sample 4 had a much larger average of macro particle size and density than did sample 3. The average size of the macro particles in sample 3 was approximately one micron with a density of 10³ particles/cm². The average size of macro particles in sample 4 was approximately three microns with a density of 10⁴ particles/cm².

### Example 2—IPD Deposited Coatings

The vacuum chamber 4, see FIG. 1, was pumped to a suitable working pressure, typically in the range of 0.1 mT to 30 mT; however, the ability of the IPD process to produce effective attachment surfaces having sustained release rates is not dependent on any specific working pressure within the range of 0.1 mT to 30 mT. Similarly, the IPD process is not dependent upon operating temperature. Typical operating temperatures are in the range of 25° C. to 200° C., but lower or higher temperatures may also be used. The temperature employed is in part be determined by the substrate. Temperatures within a range between about 20° to about 40° C. are suitable for producing most attachment surfaces.

The substrate can be rotated using, for example, a turntable, or rolled past the deposition area in any orientation relative to the trajectory of the incoming deposition material. Power is supplied to the target to generate an electric arc at the target. The power can range from a few amps to several hundred amps at a voltage appropriate for the source material. Voltage is typically in the range of 12 to 60 volts and is appropriately scaled to the size of the source material which can range from a few inches to several feet in length.

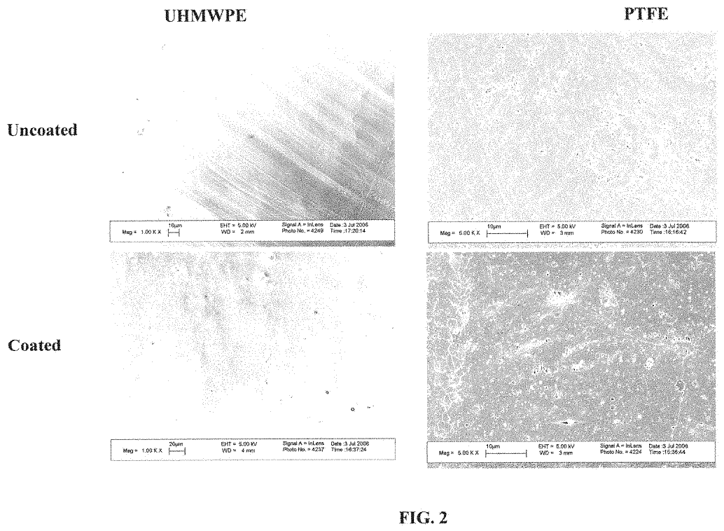

An exemplary coating of IPD deposited titanium on a UHMWPE and PTFE substrates is shown in FIG. 2. As can be seen from the SEM photographs, the deposited metal changes the surface texture to a more nano-rough surface.

Nitinol Coating on Steel

A nitinol target was placed in the vacuum chamber of the ionic plasma deposition device along with a selected substrate. The electric arc ionized the nitinol metal target into a plasma of nitinol ions, neutrally charged particles and electrons. The nitinol particles could be controlled to have a particle size ranging from less than 1 nanometer to about 50 microns.

The nitinol target is preferably medical grade. High purity target material is recommended in order to avoid potentially toxic impurities, although in some cases satisfactory results may be obtained with metals of lower purity. Different alloys can also be used; e.g., CoCrMo.

Using the described deposition process, a custom nitinol surface was deposited onto a steel substrate. A nickel and titanium, target was used with equal power to create a 50/50 mix of nickel/titanium. This mixture was deposited onto a steel coupon and analyzed by SEM and EDX. The SEM scan showed the average size of the macro particles in the sample was approximately one micron with a density of 10⁴ particles/cm². The EDX showed about 51% titanium, 49% nickel mixture evenly distributed on the surface. A standard pull test showed greater than 1 KSI (1000 psi) of adhesion strength.

Gold Coating on Nitinol

Using the disclosed deposition process, a five micron coating of gold was deposited onto a commercially available ⅛ in diameter by 0.005 in thick wall Nitinol tube. This seed layer was analyzed by SEM. The SEM scan showed an average macro particle size of approximately one micron with a density of approximately 10⁴/cm². A standard pull test showed greater than 1 KSI (1000 psi) of adhesion strength.

Titanium Seed Layer on Al₂O₃

An Al₂O₃ disk was coated with three microns of titanium as a seed layer using the deposition process of Examples 1 and 2. This seed layer was analyzed by SEM. The SEM scan showed the average size of the macro particles in the sample was approximately one micron with a density of 10⁴ particles/cm². A standard pull test showed greater than 1 KSI (1000 psi) of adhesion strength.

In a further step, titanium was flame sprayed on the seed layer and another pull test was performed. Again, the coating showed a strength of greater than 1 KSI.

Nitinol Coating on Stent

Nitinol was deposited on a stent using the disclosed deposition process. The coating was deposited to a thickness of 1 micron with an average macro particle size of one micron and a density of 10⁴ particles/cm². A standard pull test showed greater than 1 KSI of adhesion strength. The coating appeared to have the necessary characteristics for vascular tissue attachment to surfaces, thereby with the expectation of inhibiting restenosis.

### Example 3-Osteoblast Adhesion on Coated Polymer Substrates

Titanium and gold coated polymer substrates were prepared. The substrates were PEEK, UHMWPE and PTFE, each coated with gold, titanium or uncoated.

All substrates were placed in 12-well tissue culture plates (Corning, N.Y.) and were rinsed with sterilized phosphate buffered saline (PBS), 1X strength, containing 8 g NaCl, 0.2 g KCl, 1.2 g. Na₂HPO₄ and 0.2 g KH₂PO₄ in 1000 ml deionized water adjusted to pH of 7.4 (all chemicals from Sigma). Osteoblasts were then seeded at a concentration of 2500 cell/cm² onto the compacts of interest in 2 ml of DMEM (Hyclone) supplemented with 10% FBS (Hyclone) and 1% P/S and were then incubated under standard cell culture conditions at 37° C., 5% CO₂ and 95% humidified air. After 4 hr, cell culture medium was aspirated from the wells and the substrates rinsed with PBS three times to remove non-adherent cells. Adherent cells were fixed with 4% formaldehyde (Fisher Scientific, Pittsburgh, Pa.) and stained with Hoechst 33258 dye (Sigma). The cell nuclei were visualized and counted under a fluorescence microscope (Leica) using excitation at 365 nm, emission at 400 nm. Cell counts were expressed as the average number of cells on eight random fields per substrate. All experiments were run in triplicate and cell adhesion was evaluated based on the mean number of adherent cells. Numerical data were analyzed using standard analysis of variance (ANOVA). Statistical significance was considered at p<0.01.

Osteoblast morphology and adhesion location on the substrates of interest were examined using SEM. At the end of the adhesion assay, cells were dehydrated through sequential washings in 50, 60, 70, 80, and 90% ethanol solutions. Samples were then sputter-coated with a thin layer of gold-palladium using a Hummer I Sputter Coater (Technics) in a 100 millitorr vacuum in an argon environment for three minutes and 10 mA of current. Similar to samples without cells, images were taken using a JEOL JSM-840 Scanning Electron Microscope at a 5 kV accelerating voltage. Digital images were recorded using the Digital Scan Generator Plus (JEOL) software.

Results showed that compared to the respective uncoated samples, osteoblast adhesion increased on the three polymer substrates (PEEK<UHMWPE and PTFE) coated with either nanoparticulate Ti or Au. Osteoblast adhesion was greater on all samples coated with nanoparticulate Ti compared with currently used micron grain size Ti.

PTFE coated with either nanoparticulate Ti or Au outperformed both PEEK and UHMWPE coated with either nanoparticulate Ti or Au, respectively. The best osteoblast adhesion was demonstrated with PTFE coated with Ti. Table 1 shows results of osteoblast incubation of uncoated substrates compared with coated substrates.

TABLE 1

| | | | | Relative | |
|---|---|---|---|---|---|
| Sample | Substrate | Coating | Number | Change | % change |
| 1 | PEEK | None | 49.6 | 1.00 | 0 |
| 2 | PEEK | Ti | 83.2 | 1.68 | 67.74 |
| 3 | PEEK | Au | 71.7 | 1.45 | 44.56 |
| 4 | PTFE | None | 70.5 | 1.00 | 0 |
| 5 | PTFE | Ti | 82.5 | 1.17 | 17.2 |
| 6 | PTFE | Au | 73 | 1.04 | 3.55 |
| 7 | UHMWPE | None | 27.3 | 1.00 | 0 |
| 8 | UHMWPE | Ti | 56.6 | 2.07 | 107.33 |
| 9 | UHMWPE | Au | 65.8 | 2.41 | 141.03 |

Cell morphology results matched those obtained quantitatively; i.e., osteoblasts showed increased cell spreading on polymers coated with either Ti or Au compared to uncoated samples.

### Example 4—Osteoblast Proliferation on Titanium Coated UHMWPE and PTFE

PTFE and UHMWPE substrates were coated with titanium as described. Uncoated PTFE and UHMWPE samples were trimmed with a razor to make a flat adhesion surface. Before seeding, the samples were either sonicated in 70% ethanol and autoclaved or exposed to ultraviolet light at 120-350 nm for 20 min. Osteoblasts (ATCC CRL11373) were grown in culture until confluence in DMEM supplemented with 10% FBS and 1% P/S.

Osteoblasts were seeded onto each substrate at 3500 cells/cm² and then placed in 12- and 24-well cell culture plates. 175 μl of cell-containing droplets in media was placed onto the samples and incubated at 37° C. in 5% CO₂ for 4 hr. Specimens were then washed 3 times with PBS, fixed in formaldehyde for 10 min, and again washed 3× in PBS. Cells were then counted using fluorescent microscopy and DAPI dye. Images of cell morphology were taken. Experiments were conducted in triplicate with two repeats each (total of six samples for each averaged data point.) Standard statistical analysis (student t-test) was used to determine differences between substrates.

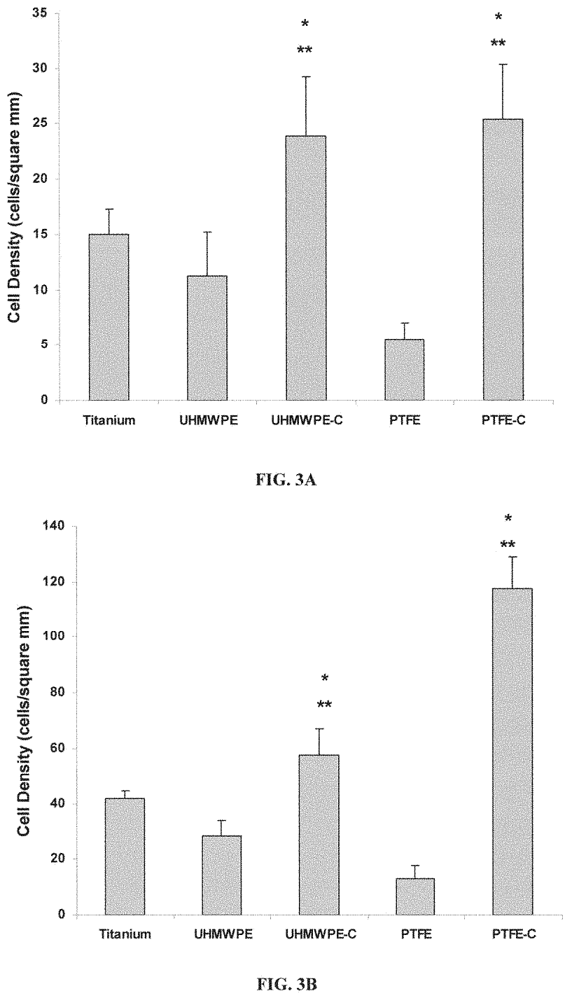

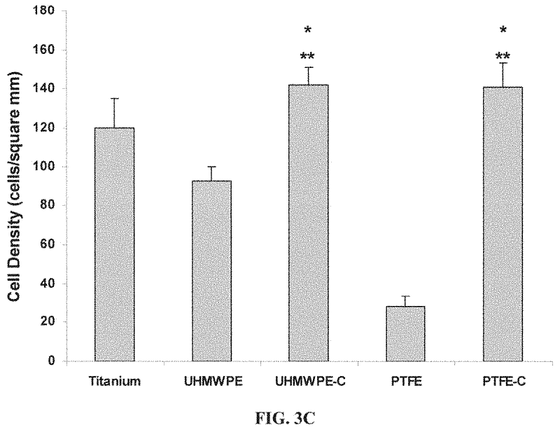

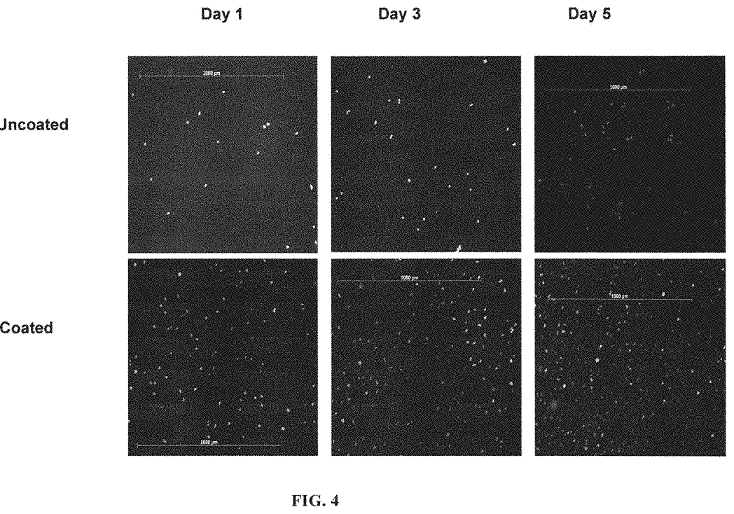

Results showed that titanium nano-surfaced coatings significantly increase proliferation of bone cells on UHMWPE and PTFE substrates compared with the corresponding uncoated samples. Statistical significance for a group of samples could not be obtained, likely because of differences in coating densities for each sample; nevertheless, the difference between each coated and uncoated sample was significant. FIG. 3A compares cell proliferation on day 1 as measured in cells per square millimeter for uncoated and titanium coated UHMWPE and PTFE; FIG. 3B for titanium coated and uncoated UHMWPE and PTFE on day 3; and FIG. 3C for titanium coated and uncoated UHMWPE and PTFE on day 5. The titanium coated UHMWPE is superior to the PTFE substrate as shown in Table 2. The increased cell osteoblast proliferation on titanium coated PTFE is initially about half of the comparative increase observed on titanium coated UHMWPE. On days 3 and 5, the titanium coated PTFE shows less than a 2-fold increase in cell proliferation compared with uncoated substrate while the titanium coated UHMWPE maintains over a 5-fold enhanced proliferation compared with its uncoated counterpart even after 5 days. Statistical analysis of the assay results for UHMWPE for N=6 had a p<0.1 compared to respective uncoated samples.

TABLE 2
Increase in cell proliferation

| Substrate | Day 1* | Day 3* | Day 5* |
|---|---|---|---|
| Ti coated | 5.3 | 8.8 | 5.8 |
| UHMWPE | | | |
| Ti coated | 2.7 | 1.9 | 1.5 |
| PTFE | | | |

*compared to corresponding uncoated substrate

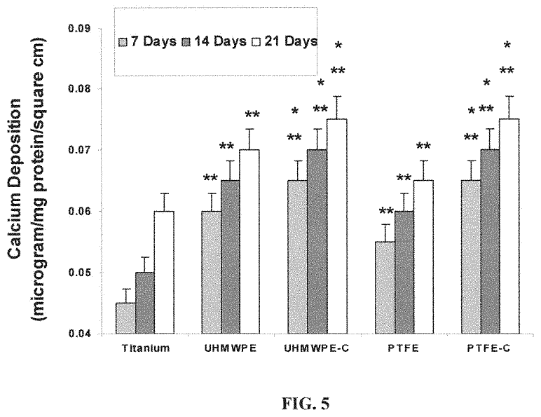

Fluorescence microscopy photographs of the proliferated cells taken at 10× magnification comparing days 1, 3 and 5 for titanium coated PTFE are shown in FIG. 4. FIG. 5 shows a comparison of the proliferated osteoblast cells at days 1, 3, and 5 on titanium coated UHMWPE.

### Example 5—Endothelial Cell Adhesion on Titanium

In this example, three types of substrates were coated with 200 nm of Ti 6-4. The average nano-particle size of the coating was 30 to 40 nanometers and was confirmed via SEM analysis.

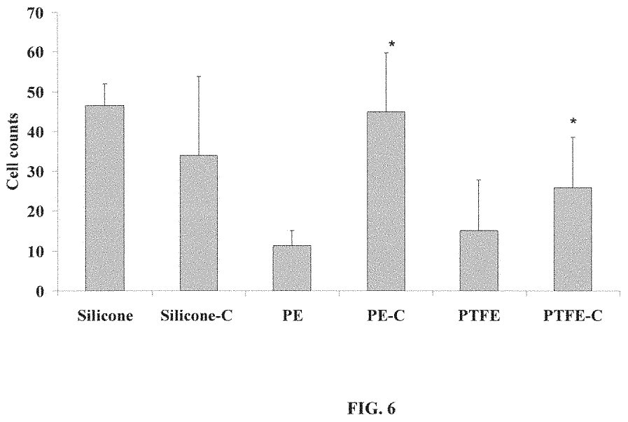

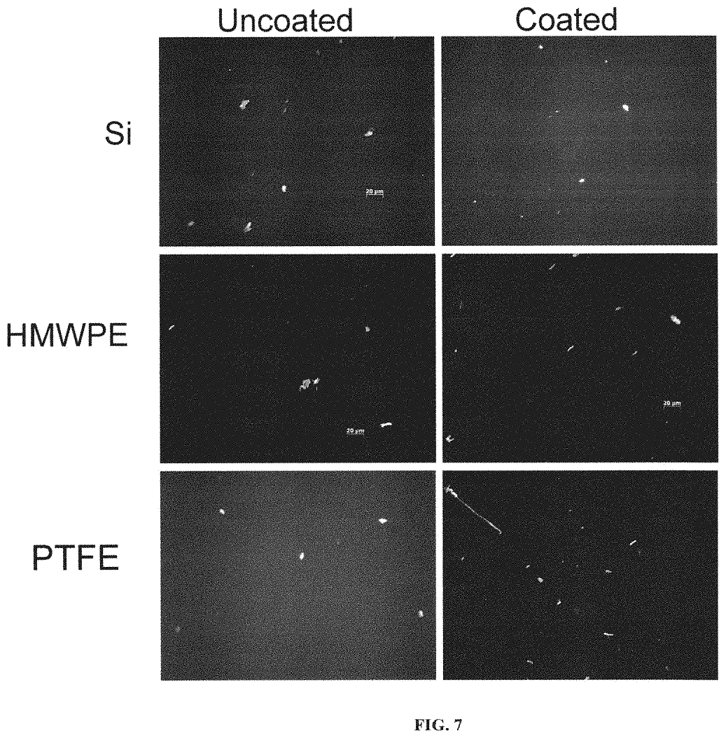

Results showed a 25% decrease in cell adhesion on the coated silicone parts, a 500% increase in cell adhesion on the coated UHMWPE and an increase of 100% cell adhesion on the PTFE samples of 100% illustrated in FIG. 6. FIG. 7 shows fluorescent microscopy images of endothelial cell density on coated and uncoated silicone, polyethylene and Teflon®.

### Example 6—Fibroblast Adhesion on Titanium Coated Substrate

Fibroblasts were seeded onto each substrate at 3500 cells/cm². The samples were placed in 12 and 24 well cell culture plates. 175 μl of cell-containing droplets in media were placed onto the samples and incubated at 37° C. and 5% CO₂ for 4 hr. At the end of the prescribed time period, specimens were washed 3 times with PBS, fixed in formaldehyde for 10 min, and again washed 3× in PBS. Cells were then counted using fluorescent microscopy and DAPI dye. Images of cell morphology were taken. Experiments were conducted in triplicate with two repeats each (total of six samples for each averaged data point.) Standard statistical analysis (student t-test) was used to determine differences between substrates.

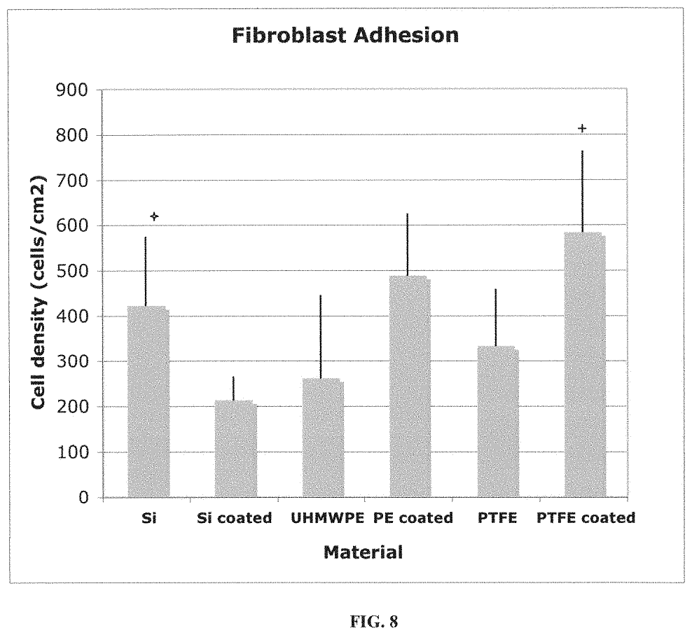

As shown from cell density measurements, fibroblast adhesion was significantly increased on PTFE and UHMWPE coated samples compared with uncoated samples, representing increases of approximately 78% and 90% respectively (FIG. 8). Increased fibroblast numbers and spreading for titanium coated UHMWPE and PTFE was also observed.

### Example 7—Fibroblast Attachment/Repulsion

In this example, three types of substrates, UHMWPE, silicone and PTFE were coated with 200 nm of Ti 6-4. The average nano-particle size of the coating was 30 to 40 nanometers and was confirmed by SEM analysis.

Fibroblasts were purchased from ATCC (CRL-2317) and grown in culture until confluence in DMEM with 10% FBS and 1% P/S. Material samples were used as supplied. Before cell experiments, samples were sonicated and autoclaved.

Fibroblasts were seeded onto each substrate at 3500 cells/cm². Samples were first placed in 12- and 24-well cell culture plates. 175 μl of cell-containing droplets in media were added into each and incubated at 37° C. under 5% CO₂ for 4 hours. Specimens were then washed 3 times with PBS, fixed in formaldehyde for 10 min, and again washed 3 times in PBS. Cells were then counted using fluorescent microscopy and DAPI dye. Cell morphology images were also acquired. Experiments were conducted in triplicate and repeated twice for each sample (total of six samples for each averaged data point). A student t-test was used to determine differences between substrates.

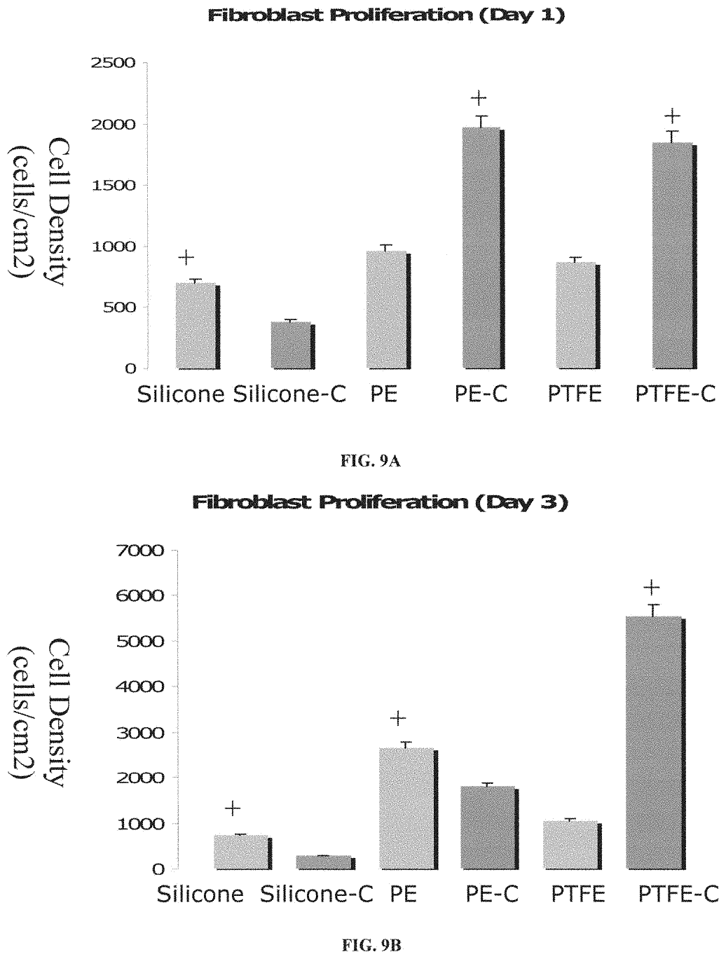

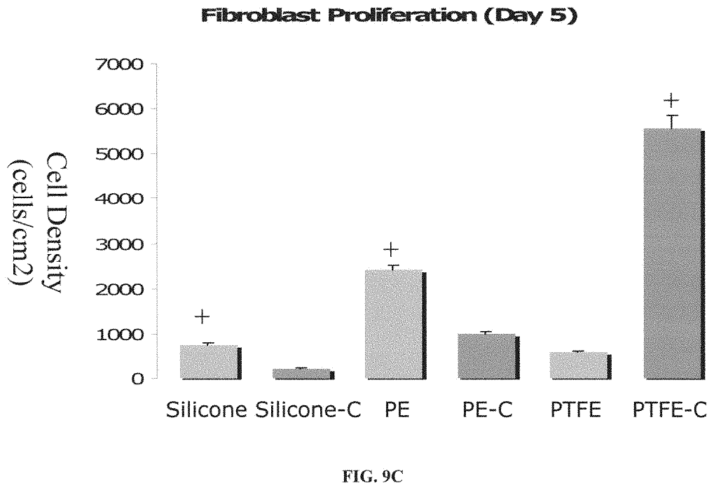

Results of this study showed for the first time that in vitro fibroblast adhesion decreased on titanium coated on silicone compared to other samples tested in this study (FIG. 8). For all other substrates, fibroblast adhesion increased on the coatings compared to uncoated samples. Fibroblast proliferation tested 1, 3, and 5 days in culture showed even more dramatic increase in fibroblast adhesion to titanium coated PTFE but less adhesion on titanium coated silicone and UHMWPE compared with the respective uncoated samples. Results for the 1, 3, and 5 day tests are shown in FIGS. 9A, 9B and 9C. Each bar represents n=3 where *p<0.01 for each comparison. This was a promising result as less adhesion of fibroblasts translates into less soft, scar tissue formation around either an orthopedic or vascular implant composed of Titanium coated on silicone. For all other substrates, fibroblast adhesion increased on the coatings compared to uncoated samples.

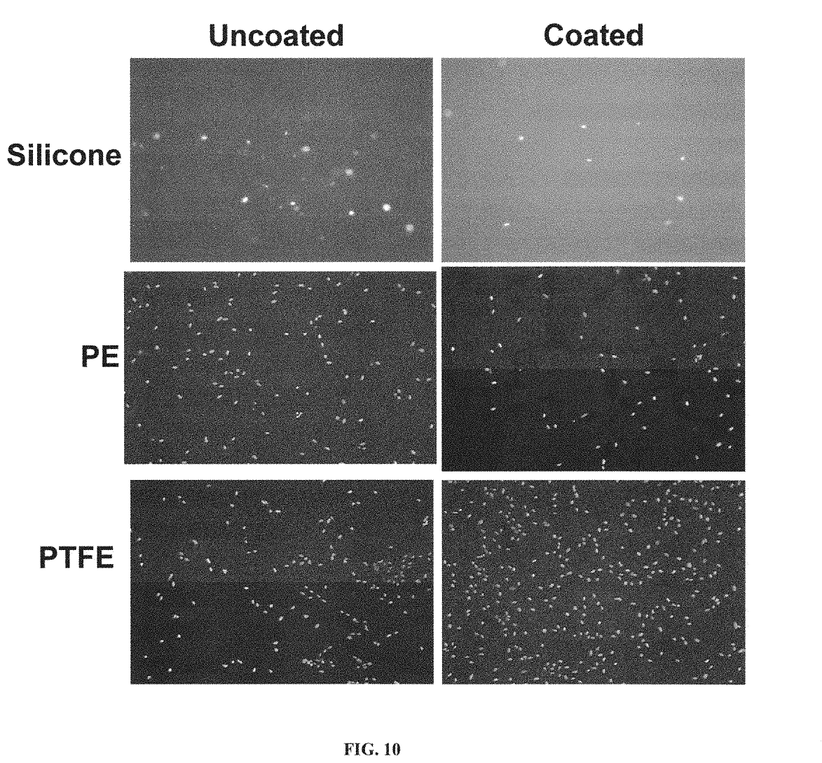

Qualitative fibroblast morphology images matched the quantitative data of less fibroblast adhesion on titanium coated silicone. Fewer well-spread cells were observed on titanium coated silicone compared to other substrates tested, as shown in FIG. 10 as analyzed by fluorescence microscopy.

### Example 8—Increased Protein Synthesis on Coated and Uncoated Samples

In this example, three types of substrates were coated with 200 nm of Ti 6-4. The average nano-particle size of the coating was 30 to 40 nanometers and was confirmed via SEM analysis. Osteoblasts were purchased from ATCC (CRL-11372) and grown to confluence in culture in DMEM with 10% FBS and 1% P/S.

Coated material samples were used as supplied. Uncoated samples were trimmed with a razor to make the adhesion surface flat. Before cell experiments, samples were either sonicated in 70% ethanol and autoclaved or UV treated for 20 minutes.

Osteoblasts were seeded onto each substrate at 3500 cells/cm². Samples were first placed in 12- and 24-well cell culture plates. 175 μl of cell-containing droplets in media was placed onto the samples and incubated at 37° C. in a 5% CO₂ atmosphere for 4 hours. The cell containing droplets were then removed and each sample well filled with DMEM media and incubated again under the same conditions for 1, 3, and 5 day proliferation. Specimens were then washed 3 times with PBS, fixed in formaldehyde for 10 min, and again washed in PBS 3 times after 24, 72, and 120 hours, respectively. Cells were then counted using fluorescent microscopy and DAPI dye. Images of cell morphology were also acquired. Experiments were conducted in triplicate and repeated twice for each sample (total of six samples for each averaged data point). A student t-test was used to determine differences between substrates.

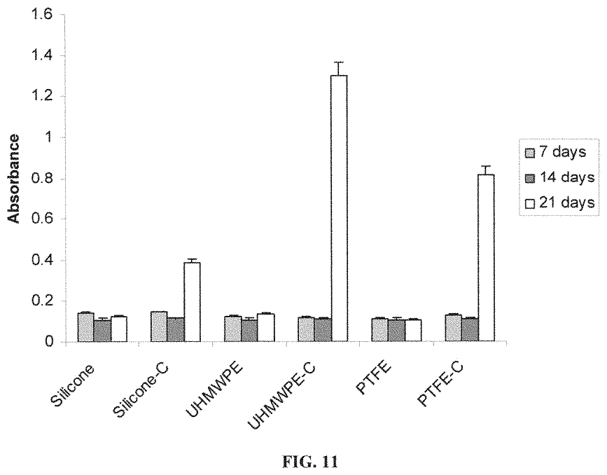

Results from protein assays showed an increase in protein synthesis for all the coated parts after 21 days. For coated silicone, the increase was approximately 400%, for coated UHMWPE, the increase was approximately 1300%, and for coated PTFE, the increase was approximately 800%. In these assays, total protein was measured. The increased proliferation at 7, 14 and 21 days is illustrated in FIG. 11.

### Example 9: Increased Osteoblast Proliferation on Silicone, PTFE and UHMWPE

In this example, three types of substrates were coated with 200 nm of Ti 6-4 through the IPD process. The average nano-particle size of the coating was 30 to 40 nanometers and was confirmed by SEM analysis.

Osteoblasts were purchased from ATCC (CRL-11372) and grown in culture until confluence in DMEM with 10% FBS and 1% P/S. Titanium coated silicone, UHMWPE and PTFE samples were used as supplied. Uncoated samples were trimmed with a razor to make the adhesion surface flat. Before cell experiments, the coated substrates were either sonicated in 70% ethanol and autoclaved or irradiated under ultraviolet light for 20 minutes.

Osteoblasts were seeded onto each substrate at 3500 cells/cm². Samples were placed in 12- and 24-well cell culture plates. 175 μl of cell-containing droplets in media was placed onto the wells and incubated at 37° C. in a 5% CO₂ atmosphere for 4 hours. The cell containing droplets were removed and each sample well filled with DMEM media and incubated again under the same conditions for 1, 3, and 5 day proliferation. Specimens were then washed 3 times with PBS, fixed in formaldehyde for 10 min, and again washed in PBS 3 times after 24, 72, and 120 hours respectively. Cells were counted using fluorescent microscopy and DAPI dye. Images of cell morphology were also acquired. Experiments were conducted in triplicate with two repeats each (total of six samples for each averaged data point). Standard statistical analysis (student t-test) was used to determine differences between substrates.

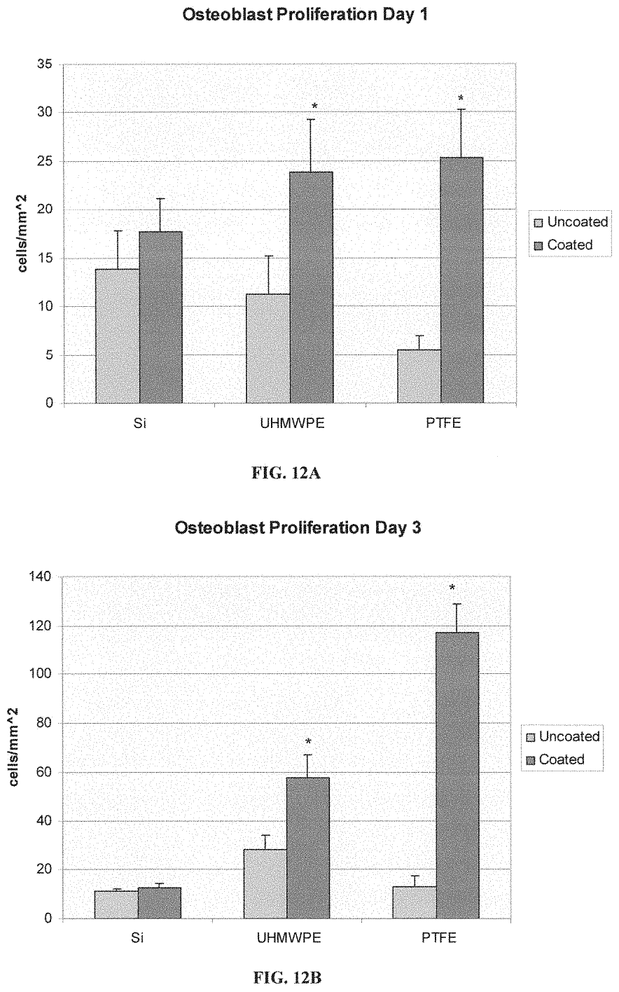

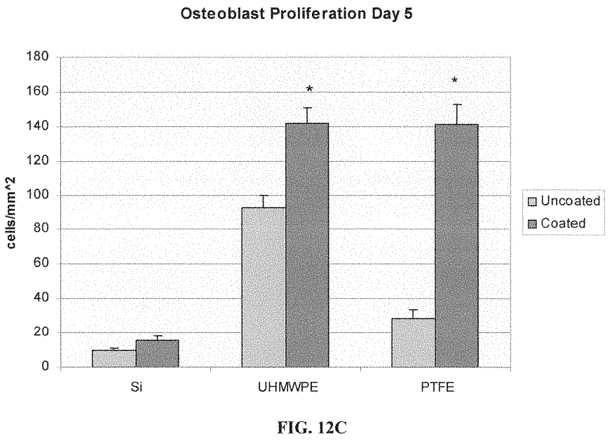

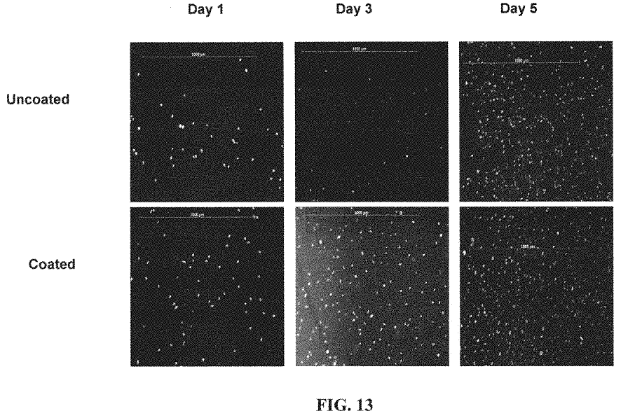

Results of the 1, 3 and 5 day test show increased osteoblast proliferation on all coated substrates over their uncoated counterparts. Cell proliferation on the coated substrates compared to uncoated substrates is shown after 1 day in FIG. 12A; after 3 days in FIG. 12B and after 5 days in FIG. 12C. FIG. 13 is a photograph of fluorescent images of DAPI stained cells on coated and uncoated PTFE for days 1, 3 and 5 on Ti coated and uncoated PTFE. There is significant cell osteoblast proliferation as early as day 1 compared with the uncoated substrates. Data are summarized in Table 3.

TABLE 3

| Substrate | Day 1 | Day 3 | Day 5 |
|---|---|---|---|
| Silicone | 25% | 10% | 25% |
| UHMWPE | 100% | 100% | 50% |
| PTFE | 400% | 1000% | 400% |

### REFERENCES

R. Furlong, J. F. Osborn, “Fixation of hip protheses by hydroxyapatite ceramic coatings,” J Bone Joint Surg (Br), 73B, 741-745, 2001.
K. C. Baker, M. A. Anderson, S. A. Oehlke, A. I. Astashkina, D. C. Haikio, J. Drelich and S. W. Donahue, “Growth, characterization and biocompatibility of bone-like calcium phosphate layers biomimetically deposited on metallic substrata,” Materials Science and Engineering: C, In Press.
T. J. Webster, J. U. Ejiofor, “Increased osteoblast adhesion on nanophase metals: Ti, Ti6Al4V, CoCrMo,” Biomaterials, 25 (19), 4731-4739, 2004.
R. Z. Valiev, V. V. Stolyarov, H. J. Rack and T. C. Lowe, “SPD-processed ultra-fine grained Ti materials for medical applications”, Medical Device Materials, ASM, Materials Park, Ohio, 362-367, 2004.
C. Yao, V. Perla, J. L. McKenzie, E. B. Slamovich, and T. J. Webster, “Anodized Ti and Ti6Al4V possessing nanometer surface features enhances osteoblast adhesion,” Journal of Biomedical Nanotechnology, 1, 68-73, 2005.
M. Sato, E. B. Slamovich and T. J. Webster, “Enhanced osteoblast adhesion on hydrothermally treated hydroxyapatite/titania/poly (lactide-co-glycolide) sol-gel titanium coatings,” Biomaterials, 26(12), 1349-1357, 2005.
K. C. Popat, E. E. L. Swan, V. Mukhatyar, K. I. Chatvanichkul, G. K. Mor, C. A. Grimes, T. A. Desai, “Influence of nanoporous alumina membranes on long-term osteoblast response”, Biomaterials, 26, 4516-4522, 2005.
Kaplan F. S, et. al., Biomaterials In Simon S P, editor. Orthopedic basic science. Columbus, Ohio: American Academy of Orthopedic Surgeons, 460-478, 1994A.
Kaplan F. S., et. al. “Form and function of bone”, In Simon S P, editor; Orthopedic basic science; Columbus, Ohio: American Academy of Orthopedic Surgeons, 127-185, 1994B

What is claimed is:

1. An article comprising:
a substrate surface; and
a nanostructure disposed on the substrate surface via ion plasma deposition (IPD), wherein the nanostructure comprises particles embedded in the substrate surface, wherein the nanostructure substrate surface enhances osteoblast proliferation compared to an uncoated substrate.

2. The article according to claim 1, wherein the substrate surface comprises one of polyether ether ketone (PEEK), ultra-high-molecular-weight polyethylene (UHMWPE), expanded polytetrafluoroethylene (EPTFE), polytetrafluoroethylene (PTFE), polypropylene, polyurethane, polyimide, polyester, and nylon.

3. The article according to claim 1, wherein the nanostructure comprises particle sizes between about 1 nanometer and about 50 microns.

4. The article according to claim 3, wherein the nanostructure comprises particle sizes between about 1 nanometer and about 100 nanometers.

5. The article according to claim 4, wherein the nanostructure comprises particle sizes of about 15 nanometers.

6. The article according to claim 1, wherein the nanostructure comprises a nanoparticle density between about 10³ particles/centimeter² and about 10⁴ particles/centimeter².

7. The article according to claim 1, wherein the nanostructure has a thickness on the substrate of between about 0.1 and about 50 microns.

8. The article according to claim 1, wherein the nanostructure is adhered to the substrate in the absence of a gas.

9. The article according to claim 1, wherein the nanostructure comprises titanium nanoparticulate.

10. The article according to claim 9, wherein osteoblast cells exhibit a greater adherence on the titanium nanoparticulate coated substrate than to an uncoated substrate after 5 days exposure to the substrate.

11. The article according to claim 10, wherein osteoblast cells exhibit greater than about 600% adherence on the titanium nanoparticulate coated substrate than to the uncoated substrate after 5 days exposure to the substrate.

12. The article according to claim 9, wherein endothelial cells exhibit a greater cell adhesion on the titanium nanoparticulate coated substrate than on an uncoated substrate after 5 days exposure to the substrate.

13. The article according to claim 12, wherein endothelial cells exhibit about 500% greater cell adhesion on the titanium nanoparticulate coated substrate than on the uncoated substrate after 5 days exposure to the substrate.

14. The article according to claim 9, wherein fibroblast cells exhibit a greater cell adhesion on the titanium nanoparticulate coated substrate than on an uncoated substrate after 5 days exposure to the substrate.

15. The article according to claim 14, wherein fibroblast cells exhibit about 90% greater cell adhesion on the titanium nanoparticulate coated substrate than on the uncoated substrate after 5 days exposure to the substrate.

16. An article comprising:
a polymer substrate surface; and
a nanostructure disposed on the substrate surface via ion plasma deposition (“IPD”), wherein the nanostructure comprises:
an adhesive film having a first density of first particles; and
a second film on top of the adhesive film, the second film having a second density of second particles, more than the first density of first particles,
wherein the nanostructured substrate surface enhances osteoblast proliferation compared to an uncoated substrate.

17. The article according to claim 16, wherein the second particles comprise macro particles.

18. The article according to claim 17, wherein the first particles comprise nanoparticles.

19. The article according to claim 17, wherein the second particles comprise blobs.

20. A method of manufacturing an article comprising the steps of:
(a) providing a polymer substrate surface; and
(b) using ion plasma deposition to deposit a nanostructure on the substrate surface wherein the nanostructured substrate surface enhances osteoblast proliferation compared to an uncoated substrate.

21. The method according to claim 20, wherein step (b) comprises the steps of:
(a) depositing an adhesive film having a first density of first particles; and
(b) depositing a second film on top of the adhesive film, the second film having a second density of second particles, more than the first density of first particles.

22. An article comprising:
a polymer substrate surface; and
a nanostructure disposed on the substrate surface, wherein the nanostructure comprises particles embedded in the substrate surface, wherein the article is formed by the method of:
using ion plasma deposition to deposit an adhesive film having a first density of first particles;
using ion plasma deposition to deposit a second film on top of the adhesive film, the second film having a second density of second particles, more than the first density of first particles,
such that the nanostructured substrate surface enhances osteoblast proliferation compared to an uncoated substrate.
